(19) United States
(12) Patent Application Publication
(10) Pub. No.: US 2019/0263583 A1
(43) Pub. Date: Aug. 29, 2019

(54) BREAST MILK COOLER

(71) Applicant: MEDELA HOLDING AG, Baar (CH)

(72) Inventors: Kathy Downie, McHenry, IL (US); Carl Huval, McHenry, IL (US); Claudia Kowanda, McHenry, IL (US)

(21) Appl. No.: 16/269,082

(22) Filed: Feb. 6, 2019

Related U.S. Application Data
(60) Provisional application No. 62/635,315, filed on Feb. 26, 2018.

(51) Int. Cl. B65D 81/38; A61M 1/06; A47J 41/00

(52) U.S. Cl. CPC B65D 81/3886; A61M 1/062; F25D 3/08; A61J 2200/72; A47J 41/0044

(57) ABSTRACT

A cooler for breast milk or other liquids is disclosed. The cooler comprehends an interior pocket adapted to receive lactation accessories that a user wishes to be cooled. The interior pocket can be adapted to be positioned in a variety of locations within the cooler to enhance or restrict the efficiency of thermal transmission between the contents of the pocket and a cooling element located within the cooler. Also disclosed is a sensor that can be associated with the pocket. The sensor can provide a temperature output to an external device or a display in or on the container delineating a temperature within the cooler.

### CROSS-REFERENCE TO RELATED APPLICATIONS

This application is the non-provisional, and claims the benefit of the filing date of, U.S. Provisional Application No. 62/635,315, filed Feb. 26, 2018, the entirety of which is hereby incorporated by reference. A design patent application, U.S. Design patent application No. 29/638,278, was filed Feb. 26, 2018 bearing title “Cooling Bag for Bottles and Accessories”, the subject matter of which is related to the present utility application, and is incorporated hereby incorporated by reference in its entirety.

### STATEMENT OF FEDERALLY SPONSORED RESEARCH AND DEVELOPMENT

None.

### FIELD OF THE DISCLOSURE

The present disclosure relates generally to portable coolers for breast milk or liquids along with cooling various accessories associated with lactation.

### BACKGROUND

Various cooling devices are well known in the art. Examples are the Medela Breast Milk Cooler Set as well as products from various other manufacturers. These coolers generally have an outer container that is insulated as well as an interior cooling element that can be removed and placed in a freezer or the like. After the interior cooling element has reached a desired temperature it is replaced in the outer container and, after breast milk is pumped from the mother, collection containers can be placed in the cooler in thermal contact with the cooling element thereby reducing the temperature of the expressed milk and allowing for extended storage of the expressed milk. Additionally, nursing mothers who experience irritation due to nursing or expressing breast milk can find relief from application of a conformable pad applied to the affected area, such as the Medela Tender Care™ hydrogel pad. The pad can be cooled, if desired, prior to use.

### SUMMARY

Therefore, it is an object of the invention to provide a portable cooling container which is effective to cool expressed milk or other liquids.

It is a further object of the invention to provide a cooling element that can be placed in contact with a breast feeding accessory

It is another object of the invention to allow a user to vary the amount of heat transfer from the accessory to the cooling element.

It is an additional object of the invention to provide with a temperature sensor so as to allow monitoring of the temperature of the cooling bag.

It is another object of the invention to provide a direct or indirect temperature readout of the cooler contents.

Various advantages of the present disclosure are described below regarding exemplary embodiments contained herein. The drawings and description herein are provided to illustrate examples of the general concepts discussed throughout the present disclosure. Numerous changes and modifications can be made, as known to those of skill in the art, without departing from the general principles set forth herein.

### BRIEF DESCRIPTION OF THE DRAWINGS

These and other features and advantages of the various exemplary embodiments disclosed herein will be better understood with respect to the following description and drawings, in which:

FIG. 1 is a front perspective view of an exemplary cooler constructed in accordance with the principles herein.

FIG. 2 plan view of the cooler of FIG. 1;

FIG. 3 is a plan view of the interior of the cooler with various elements included;

FIG. 4 is partial view of the cooler and an exemplary external device; and

FIGS. 5A and 5B illustrate exemplary displays.

Common reference numerals are used throughout the drawings and the detailed description to indicate the same elements.

### DETAILED DESCRIPTION

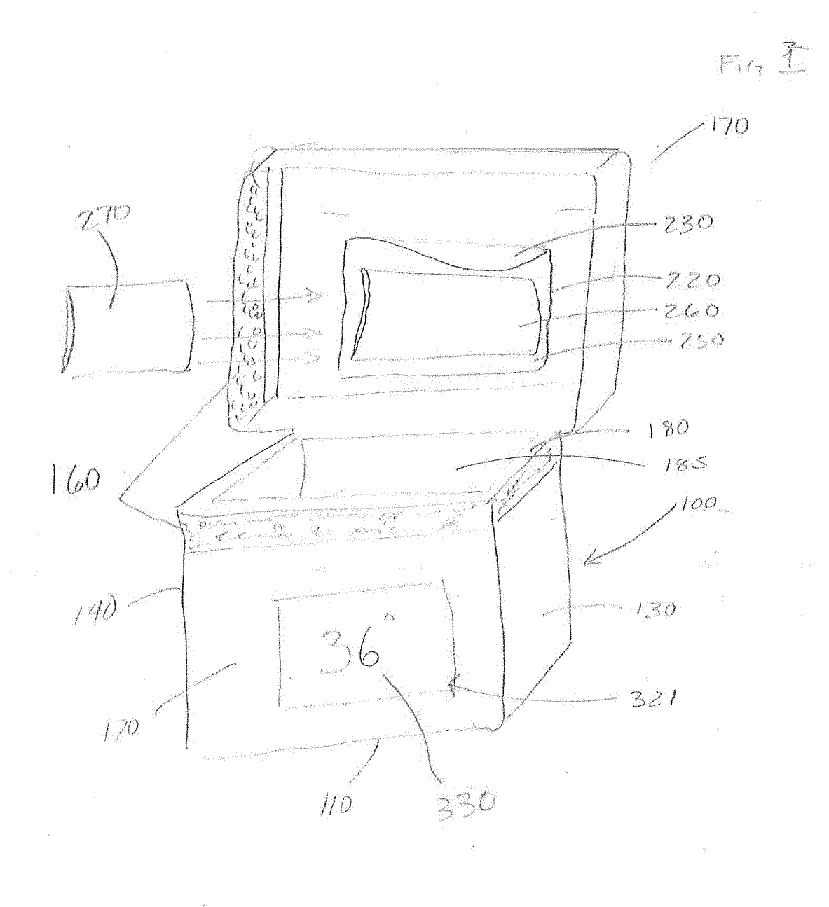

As shown in FIG. 1, the present invention comprehends a bottle container, or cooler, [100] having a bottom [110], a first exterior wall, [120] and second exterior wall [130], a third exterior wall [140] and a fourth exterior wall [150]. Each of the walls is surmounted by a closure [160] which can be removably affixed to a container top [170]. The closure [160] can be a zipper, a hook and loop closure or some other type of suitable closure. Additionally, the closure [160] can surmount three of the four walls with the fourth wall extending onto the top [170] so as to form a hinge therewith.

Interior to the walls [120,130,140,150] bottom [110] and top [170] is placed insulation [180] which is operative to thermally isolate the interior of the container [100] from the exterior environment. Interior to the insulation [180], the container [100] is lined with a non-absorbent material [185] to facilitate cleaning the container [100].

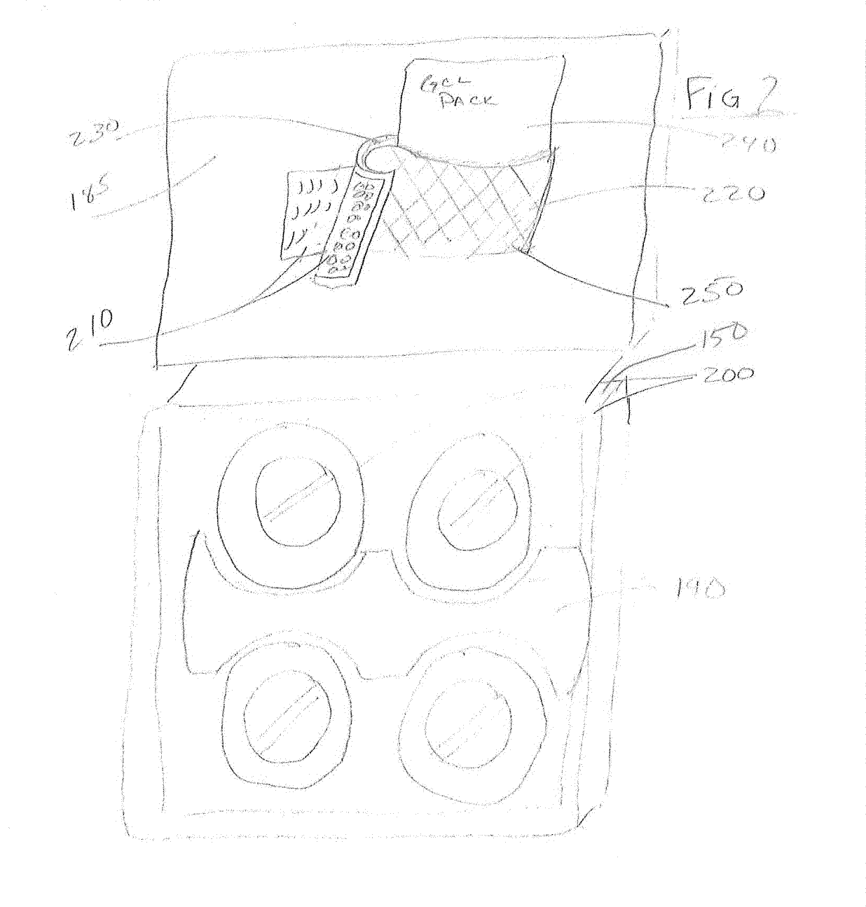

As shown in FIG. 2, the cooler can further include a cooling element [190] which can be removed from the container [100] and cooled in a freezer or the like. The cooling element [190] can be configured to accept a plurality of receptacles [200] for expressed milk or other liquids.

Referring to FIG. 2, the top [170] can include an interior fastener [210] which allows a pocket [220] to be removably affixed to the liner [185]. The pocket [220] defines a receiver [230] which can receive a lactation accessory, such as a hydrogel pack [240]. The pocket can be formed of one or more suitable materials, such as mesh, vinyl, or any other material(s) that can retain the accessory in the pocket. The pocket can include one or more expandable portions formed using elastic or other material, for example. Further, the pocket can be formed of a thermochromic material or contain thermochromic printing or indicator thereon. Additionally, the pocket defines an interior face [250] which is adapted to provide efficient heat transfer from the accessory [240] to the cooling element [190]. Alternatively, the pocket [220] can be sewn or otherwise secured to a particular location of the liner [185]. However, there are times when a user does not wish the accessory [240] to be cooled to the extent that direct or indirect contact with the cooling element [190] provides. In those situations, the interior face [250] additionally has a sleeve [260] defined thereon. The sleeve [260] is adapted to receive a removable insulating element [270] which is operative to reduce the rate of heat transfer between the accessory [240] and the cooling element [190]. The insulating element can be formed of material, or can be a printed on paper or cardboard or similar material and provided within the lactation accessory packaging, if desired.

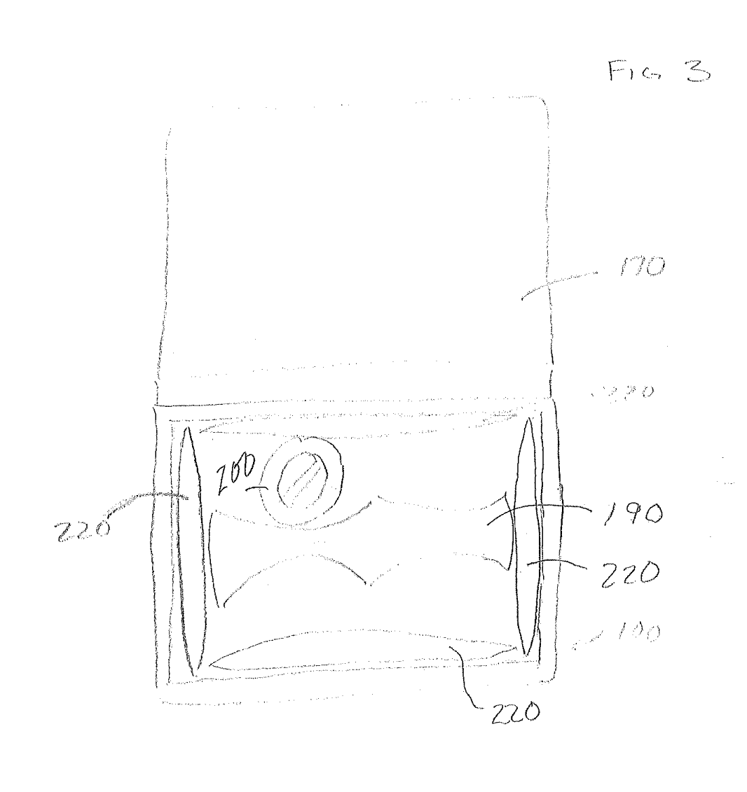

As shown in FIG. 3, the pocket [220] can be removed from the top [170] and placed at differing locations within the container [100]. This facilitates greater choice on the part of the user to configure the location of the pocket [220] relative to the cooling element [190] or the receptacles [200].

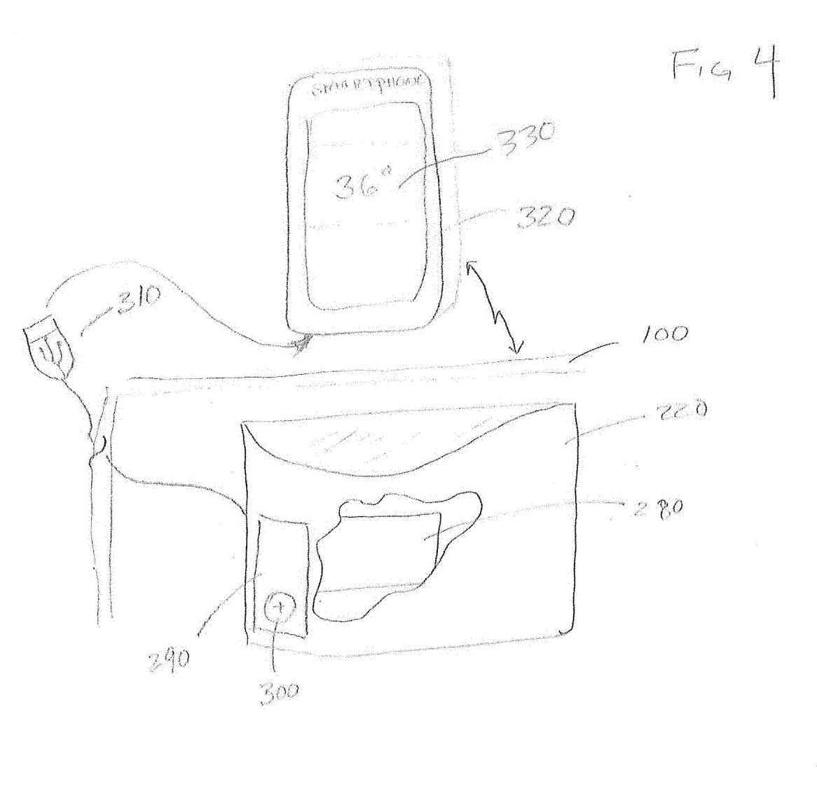

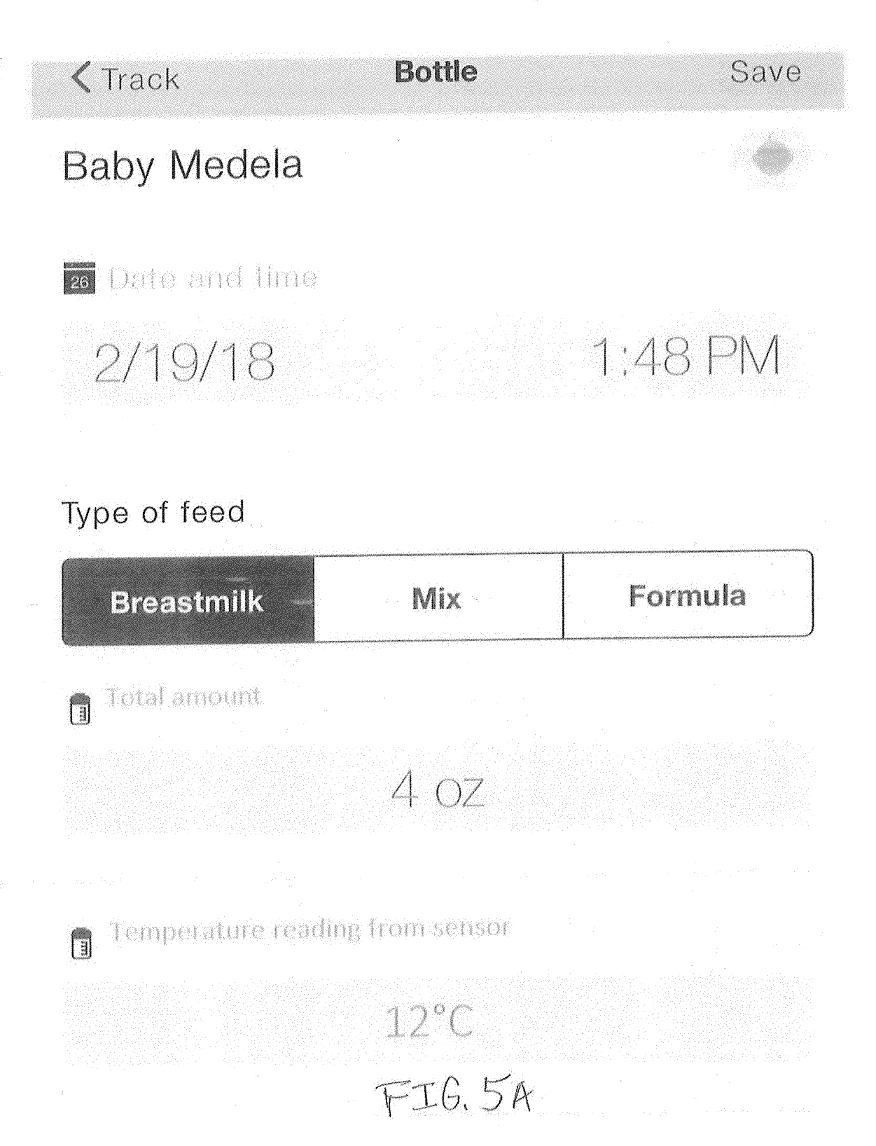

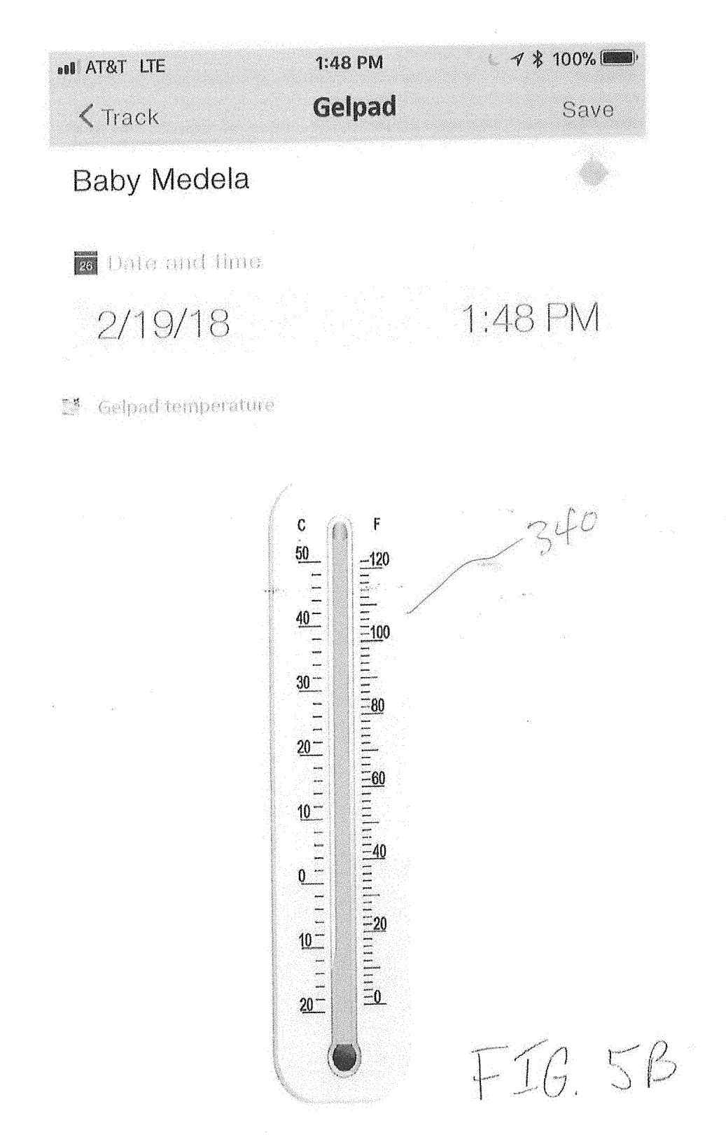

In another embodiment, best shown in FIGS. 4, 5A and 5B, the pocket [220] comprehends a suitable temperature or other sensor [280] which is operative to sense the temperature of the container [100] interior, and/or other parameter(s) of the container. The sensor [280] can be operatively connected to the requisite electronics [290] and a power source, such as a battery, solar panel, or the like [300], if needed, to render the sensor operational, or can otherwise provide an output for display on an internal or external device. Dependent on the location of the pocket [220] within the container [100] the sensor can also determine the temperature of items in contact with the pocket, such as the cooling element [190] or the receptacles [200] or the general temperature of the container interior. The sensor can be connected to or provide an output [310] which is capable of being sent and received by an external device [320], or a display on the interior or exterior of the container [321], which can provide either a numerical display [330] or a graphical display [340] of the data. The output [310] can be either a graphical output, wired output, such as one of the family of USB ports, or wireless using a protocol such as Bluetooth and the like.

Variations of the specific configurations shown and described herein that provide common functionality within the scope of the principles of the present disclosure, are contemplated to be included in all claims deriving therefrom, at least some of which are set forth herein below.

We claim:

1. A portable cooler adapted to receive a plurality of receptacles and a cooling element, the cooler comprising:
an interior;
a pocket, locatable within said cooler, said pocket adapted to receive an accessory to be thermally coupled to said interior.

2. The cooler according to claim 1 and said pocket further comprising an interior face and a sleeve defined on said interior face; said sleeve being adapted to receive an insulating element placeable between said pocket and said interior.

3. A portable cooler adapted to receive a plurality of receptacles and a cooling element, wherein said cooler defines an interior thereof, said cooler comprising:
a pocket locatable within said interior and;
said pocket being releasably affixable to a surface within said interior.

4. A portable cooler adapted to receive a plurality of receptacles, a cooling element and an accessory pocket locatable on an interior of said cooler, said pocket comprising:
a sensor associated with said pocket;
said sensor having requisite electronics operationally connected thereto and;
an output to an environment exterior to said cooler capable of transmitting data generated by said sensor to a device located in said exterior environment.

5. The sensor according to claim 4 and said sensor further comprising a temperature sensor.
